(19) United States
(12) Patent Application Publication
(10) Pub. No.: US 2024/0342171 A1
(43) Pub. Date: Oct. 17, 2024

(54) DISCOVERY OF A F-ATP SYNTHASE INHIBITOR FOR THE TREATMENT OF MYCOBACTERIUM ABSCESSUS DISEASES

(71) Applicant: Nanyang Technological University, Singapore (SG)

(72) Inventors: Gerhard GRÜBER, Singapore (SG); Roderick Wayland BATES, Singapore (SG); Priya RAGUNATHAN, Singapore (SG)

(21) Appl. No.: 18/294,472

(22) PCT Filed: Aug. 16, 2022
(86) PCT No.: PCT/SG2022/050581

§ 371 Date: Feb. 1, 2024

(30) Foreign Application Priority Data
Aug. 17, 2021 (SG) 10202108980T
Oct. 29, 2021 (SG) 10202112053Y

(51) Int. Cl. A61K 31/505; A61K 31/498; A61K 45/06; A61P 31/00

(52) U.S. Cl. CPC A61K 31/505; A61K 31/498; A61K 45/06; A61P 31/00

(57) ABSTRACT

Disclosed herein is the use of a compound of formula I, or a salt or a solvate thereof, in the treatment of a bacterial infection caused by Mycobacterium abscessus, where the compound of formula (I) has the following formula: I where: R₁, Rₐ, R₂, R₃, X, Y, Z and R₄ are as defined herein. Also disclosed herein are combination treatments using the compound of formula I and pharmaceutical formulations comprising said compound.

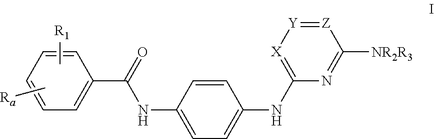

### FIELD OF INVENTION

The current invention relates to the use of compounds for the treatment of diseases associated with Mycobacterium abscessus infections.

### BACKGROUND

The listing or discussion of a prior-published document in this specification should not necessarily be taken as an acknowledgement that the document is part of the state of the art or is common general knowledge.

Mycobacterium abscessus (Mab) complex is a group of rapidly growing nontuberculous mycobacteria (NTM) that can cause severe human diseases, including respiratory, skin, and soft tissue disorders, particularly in cystic fibrosis and elderly patients. NTM infections are increasing in their global prevalence, morbidity and mortality, and frequently surpass the global incidence of TB infections in developed countries. Mab is the major NTM pathogen in Singapore and is responsible for about 80% of all pulmonary infections caused by rapidly growing NTM. Mab has been classified into the three subspecies Mycobacterium abscessus subsp. abscessus (sensu stricto), Mycobacterium abscessus subsp. massiliense, and Mycobacterium abscessus subsp. bolletii. They have different susceptibilities to antimicrobial agents, and M. abscessus subsp. abscessus is the most prevalent and resistant and is called a “nightmare” bacterium. Mab treatment is largely empirical, including a regimen of amikacin, cefocetin, clarithromycin, and imipenem for the initial phase and oral antimicrobials for the continuation phase, for a total duration of 18 to 24 months. Even with such a complex regimen and long duration, the outcome of Mab disease is still poor (40-50%), going along with severe drug-related toxicities and side-effects. In addition, most of the drugs are not bactericidal.

Mab is notoriously resistant to standard anti-TB agents and most antimicrobial agents. The pathogen is also resistant to disinfectants and, therefore, can cause postsurgical and postprocedural infections. The treatment of diseases caused by NTM, including the fast-growing bacterium Mycobacterium abscessus (M. abscessus), is problematic because of low permeability of the cell wall, biofilm formation, deficient drug-activating enzymes, numerous enzymes that neutralize drugs or modify their specific targets, naturally occurring polymorphism, and induction of drug efflux pumps. Such pumps use either Adenosine triphosphate (ATP)- or proton-motive force-driven energy forms generated by the electron transport chain (ETC), which generates the proton-motive force for condensing adenosine diphosphate (ADP) plus Pi to form ATP in the process of oxidative phosphorylation by the F₁F₀-ATP synthase. Oxidative phosphorylation is the major process for ATP synthesis in the pathogen, making the enzymes of the ETC and the F₁F₀-ATP synthase attractive as drug targets, as shown in clofazimine (CFZ) affecting NADH dehydrogenase (NDH-2) of the ETC, or the repurposed antituberculosis (TB) drug bedaquiline (BDQ) and its derivative, TBAJ876, targeting M. abscessus's F₁F₀ ATP synthase. However, despite being potent growth inhibitors, these compounds are mostly bacteriostatic against M. abscessus.

Therefore, there exists a need to discover new F-ATP synthase inhibitors for the treatment of M. abscessus diseases.

### SUMMARY OF INVENTION

It has been surprisingly found that a number of compounds can treat M. abscessus diseases. Aspects and embodiments of the invention will now be discussed be reference to the following numbered clauses.

1. Use of a compound of formula I:

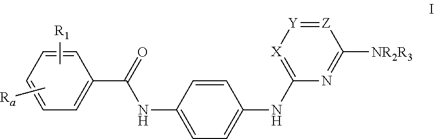

where:
R₁ and Rₐ each independently represent H, halogen, C₁-C₄ alkyl, C₁-C₄ alkoxy, or OH;
R₂ represents H, C₁-C₄ alkyl that is unsubstituted or substituted by a hydroxyl group, or —CH₂CO₂Et;
R₃ represents H or C₁-C₄ alkyl;
X, Y and Z each independently represent CR₄ or N;
each R₄ independently represents H, halogen, C₁-C₄ alkyl, or C₁-C₄ alkoxy, or a salt or a solvate thereof, in the preparation of a medicament for the treatment of a bacterial infection caused by Mycobacterium abscessus.

2. A compound of formula I, or a salt or solvate thereof, as described in Clause 1 for use in the treatment of a bacterial infection caused by Mycobacterium abscessus.

3. A method of treatment of a bacterial infection caused by Mycobacterium abscessus comprising the steps of providing a pharmaceutically effective amount of a compound of formula I, or a salt or solvate thereof, as described in Clause 1 to a subject in need thereof.

4. The use according to Clause 1, the compound for use according to Clause 2 or the method according to Clause 3, wherein the compound of formula I is one in which R₂ represents H, C₁-C₄ alkyl that is unsubstituted or substituted by a hydroxyl group.

5. The use according to Clause 1 or Clause 4, the compound for use according to Clause 2 or Clause 4 or the method according to Clause 3 or Clause 4, wherein R₃ represents H, CH₃ or CH₂CH₃.

6. The use according to Clause 1 or Clauses 4 to 5, the compound for use according to Clause 2 or Clauses 4 to 5 or the method according to Clauses 3 to 5, wherein Rₐ is H and R₁ is Br.

7. The use according to Clause 1 or Clauses 4 to 6, the compound for use according to Clause 2 or Clauses 4 to 6 or the method according to Clauses 3 to 6, wherein X is N.

8. The use according to Clause 1 or Clauses 4 to 7, the compound for use according to Clause 2 or Clauses 4 to 7 or the method according to Clauses 3 to 7, wherein Z is N.

9. The use according to Clause 1 or Clauses 4 to 8, the compound for use according to Clause 2 or Clauses 4 to 8 or the method according to Clauses 3 to 8, wherein Y is C—CH₃.

10. The use according to Clause 1 or Clauses 4 to 9, the compound for use according to Clause 2 or Clauses 4 to 9 or the method according to Clauses 3 to 9, wherein the compound of formula I, or a salt or solvate thereof, is selected from the list:

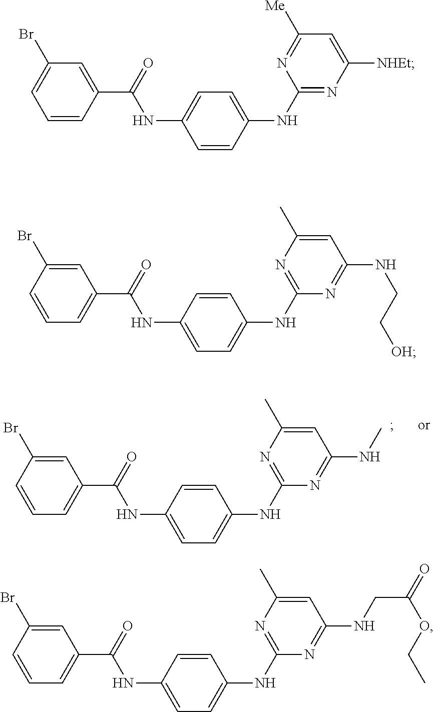

optionally wherein the compound of formula I, or a salt or solvate thereof, is selected from the list:

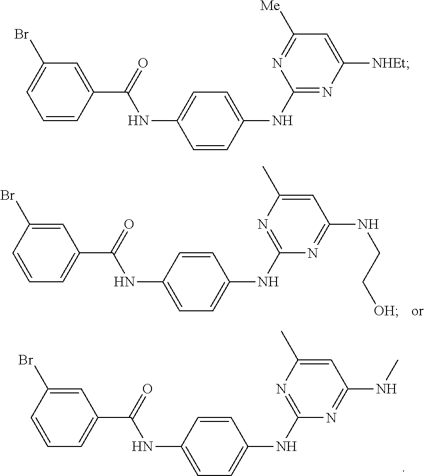

11. The use according to Clause 1 or Clauses 4 to 10, the compound for use according to Clause 2 or Clauses 4 to 10 or the method according to Clauses 3 to 10, wherein the bacterial infection is presented in the form of one or more of a respiratory, a skin or a soft tissue disorder (e.g. one or more of the group selected from a pulmonary infection, amyotrophic lateral sclerosis and bronchiectasis).

12. Use of a compound of formula I as defined in any one of Clauses 1 and 4 to 10 or a salt or a solvate thereof, and another therapeutic agent, or a salt or solvate thereof, for the preparation of a medicament for the treatment of a bacterial infection caused by Mycobacterium abscessus, wherein the compound of formula I is administered sequentially, simultaneously or concomitantly with the other therapeutic agent.

13. A compound of formula I, as defined in any one of Clauses 1 and 4 to 10 or a salt or a solvate thereof, for use in the treatment of a bacterial infection caused by Mycobacterium abscessus, wherein the compound of formula I is administered sequentially, simultaneously or concomitantly with another therapeutic agent, or a salt or solvate thereof.

14. A method of treatment of a bacterial infection caused by Mycobacterium abscessus, which method comprises the administration of a pharmaceutically effective amount of a compound of formula I as defined in any one of Clauses 1 and 4 to 10 or a salt or a solvate thereof, and another therapeutic agent, or a salt or solvate thereof, to a patient in need of such treatment.

15. The use according to Clause 12, the compound for use according to Clause 13 or the method according to Clause 14, wherein the another therapeutic agent is selected from one or more of the group consisting of an oral antimicrobial agent, amikacin, cefocetin, clarithromycin, clofazimine, and imipenem, optionally wherein the another therapeutic agent is clofazimine.

16. A pharmaceutical composition comprising a compound of formula I as defined in any one of Clauses 1 and 4 to 10 or a salt or a solvate thereof, and another therapeutic agent, or a salt or solvate thereof, wherein the another therapeutic agent is selected from one or more of the group consisting of an oral antimicrobial agent, amikacin, cefocetin, clarithromycin, clofazimine, and imipenem, optionally wherein the another therapeutic agent is clofazimine.

### DRAWINGS

FIG. 1 depicts GaMF1 susceptibility testing. (A) Structure of GaMF1; (B) Effects of GaMF1 on M. abscessus subsp. abscessus ATCC19977 (M. abscessus) growth when grown on cation-adjusted Mueller-Hinton (CAMH) medium. The experiments were performed in triplicate; (C) Effect of GaMF1 against the clinical isolate M. abscessus Bamboo in CAMH broth. The experiment was performed in triplicate. P<0.0001. Statistical analysis was carried out for the experiments in panels B to D using one-sample t and Wilcoxon tests (Motulsky, H. & Christopoulos, A., 2003. Fitting Models to Biological Data Using Linear and Nonlinear Regression: A Practical Guide to Curve Fitting. GraphPad Software Inc., San Diego CA); and (D) Six days of GaMF1 kill kinetics against M. abscessus. The bacteria were grown in liquid culture (CAMH medium) in the presence of the indicated concentrations of GaMF1. The experiments were repeated twice, and the profiles were similar. P<0.001. Statistical analysis was carried out for the experiment using ordinary one-way analysis of variance (ANOVA) with Bartlett's test.

FIG. 2 depicts GaMF1 susceptibility testing in 7H9 medium. (A) Effects of GaMF1 on the three Mab subspecies (M. abscessus subsp. abscessus, M. bolletii, and M. massiliense) growth when grown in the Middlebrook 7H9 broth. The experiments have been performed in triplicate; (B) Comparison with the clinical isolate M. abscessus Bamboo on 7H9 medium. ***: P<0.0001, statistical analysis was carried out for the experiments A-B using one sample t- and Wilcoxon test (Motulsky, H. & Christopoulos, A., 2003. Fitting Models to Biological Data Using Linear and Nonlinear Regression: A Practical Guide to Curve Fitting. GraphPad Software Inc., San Diego CA); (C) Initial three days of GaMF1 kill kinetics against M. abscessus subsp. abscessus growth. The bacteria were grown in liquid culture (7H9) in the presence of the indicated concentrations of GaMF1. The experiments were repeated twice, and the profiles were similar. ****: P<0.0001, statistical analysis was carried out using ordinary one way ANOVA Bartlett's test.

FIG. 3 depicts that GaMF1 inhibits oxidative phosphorylation. (A) Inhibition of intracellular ATP synthesis of M. abscessus cells by GaMF1. The ATP content of the cells was measured by adding BacTiter-Glo (Promega) to the cells. The total ATP content is directly proportional to number of relative luminescence units (RLU). The experiments were performed in triplicate; and (B) Inhibition of ATP synthesis by GaMF1 in M. abscessus inside-out membrane vesicles (IMVs) using the electron donor nicotinamide adenine dinucleotide (NADH). ATP synthesis by M. abscessus IMVs was measured as luminescence upon addition of CellTiter-Glo (Promega). The experiments were performed in triplicate. P<0.0001. Statistical analysis was carried out for both experiments in panels A and B using one-sample t and Wilcoxon tests (Motulsky, H. & Christopoulos, A., 2003. Fitting Models to Biological Data Using Linear and Nonlinear Regression: A Practical Guide to Curve Fitting. GraphPad Software Inc., San Diego CA).

FIG. 4 depicts the increased potency of GaMF1 in combination with the antibiotics CFZ, rifabutin, and amikacin in 7H9 medium. (A) M. abscessus growth inhibition by GaMF1 (circle) in combination with 3.4 mM (inverted triangle), 6.8 mM (open square), or 27.2 μM (open circle) CFZ; (B) GaMF1 (circle) susceptibility of M. abscessus in combination with 1 μM (square) or 4.2 μM (triangle) rifabutin; and (C) Amikacin (5 μM [square]) increases the potency of GaMF1 growth inhibition of M. abscessus subsp. abscessus. P<0.0001. Statistical analysis was carried out using two-way ANOVA for all the experiments presented.

FIG. 5 depicts the GaMF1 and rifabutin killing potency. Initial six days of GaMF1 and GaMF1 plus rifabutin kill kinetics against M. abscessus subsp. abscessus growth. The bacteria were grown in liquid culture (7H9) in the presence of the indicated concentrations of GaMF1 and rifabutin. The experiments were repeated twice, and the profiles were similar. P<0.0001. Statistical analysis was carried out for the experiment using ordinary one-way ANOVA with Bartlett's test.

FIG. 6 depicts that the F₁F_{O} ATP synthase is a molecular engine composed of the F_{O} motor (a:C₉), the F₁-engine (α₃:β₃:γ:δ:ε) and the peripheral stalk (b-δ:b′).

### DESCRIPTION

As noted above, the current invention relates to the discovery of a set of compounds that can treat infections caused by Mycobacterium abscessus.

Thus, in a first aspect of the invention there is provided a use of a compound of formula I:

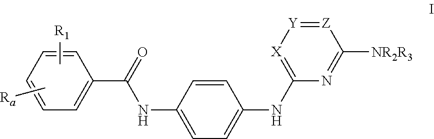

where:
R₁ and Rₐ each independently represent H, halogen, C₁-C₄ alkyl, C₁-C₄ alkoxy, or OH;
R₂ represents H, C₁-C₄ alkyl that is unsubstituted or substituted by a hydroxyl group, or —CH₂CO₂Et;
R₃ represents H or C₁-C₄ alkyl;
X, Y and Z each independently represent CR₄ or N;
each R₄ independently represents H, halogen, C₁-C₄ alkyl, or C₁-C₄ alkoxy, or a salt or a solvate thereof, in the preparation of a medicament for the treatment of a bacterial infection caused by Mycobacterium abscessus.

In a second aspect of the invention, there is provided a compound of formula I, or a salt or solvate thereof, as described above for use in the treatment of a bacterial infection caused by Mycobacterium abscessus.

In a third aspect of the invention, there is provided a method of treatment of a bacterial infection caused by Mycobacterium abscessus comprising the steps of providing a pharmaceutically effective amount of a compound of formula I, or a salt or solvate thereof, as described above to a subject in need thereof.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

References herein (in any aspect or embodiment of the invention) to compounds of formula I includes references to such compounds per se, to tautomers of such compounds, as well as to pharmaceutically acceptable salts or solvates of such compounds.

Pharmaceutically acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula I are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

Compounds of formula I, as well as pharmaceutically acceptable salt and solvates of such compounds are, for the sake of brevity, hereinafter referred to together as the “compounds of formula l”.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention.

Compounds of formula I may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

The term “halo”, when used herein, includes references to fluoro, chloro, bromo and iodo.

Unless otherwise stated, the term “alkyl” refers to an unbranched or branched, acyclic or cyclic, saturated or unsaturated (so forming, for example, an alkenyl or alkynyl) hydrocarbyl radical, which may be substituted or unsubstituted (with, for example, one or more halo atoms). Where the term “alkyl” refers to an acyclic group, it is preferably C₁₋₄ alkyl (such as ethyl, propyl, (e.g. n-propyl or isopropyl), butyl (e.g. branched or unbranched butyl), or, more preferably, methyl).

Where the term “alkyl” is a cyclic group (which may be where the group “cycloalkyl” is specified), it is preferably C₃₋₄ cycloalkyl.

Further embodiments of the invention that may be mentioned include those in which the compound of formula I is isotopically labelled. However, other, particular embodiments of the invention that may be mentioned include those in which the compound of formula I is not isotopically labelled.

The term “isotopically labelled”, when used herein includes references to compounds of formula I in which there is a non-natural isotope (or a non-natural distribution of isotopes) at one or more positions in the compound. References herein to “one or more positions in the compound” will be understood by those skilled in the art to refer to one or more of the atoms of the compound of formula I. Thus, the term “isotopically labelled” includes references to compounds of formula I that are isotopically enriched at one or more positions in the compound.

The isotopic labelling or enrichment of the compound of formula I may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷Cl, ⁷⁷Br, ⁸²Br and ¹²⁵I).

When the compound of formula I is labelled or enriched with a radioactive or nonradioactive isotope, compounds of formula I that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

The following embodiments equally relate to the use according to the first aspect of the invention, the compound for use according to the second aspect of the invention and the method of treatment according to the third aspect of the invention and may be combined with said aspects in any technically sensible manner.

Thus, in embodiments of the invention the compound of formula I, or a salt or solvate thereof, may be one in which:
(a) R₂ represents H, C₁-C₄ alkyl that is unsubstituted or substituted by a hydroxyl group;
(b) R₃ may represent H, CH₃ or CH₂CH₃;
(c) Rₐ may be H and R₁ may be Br;
(d) X may be N;
(e) Z may be N;
(f) Y may be C—CH₃.

In further embodiments of the invention that may be mentioned herein, the compound of formula I, or a salt or solvate thereof, may be selected from the list:

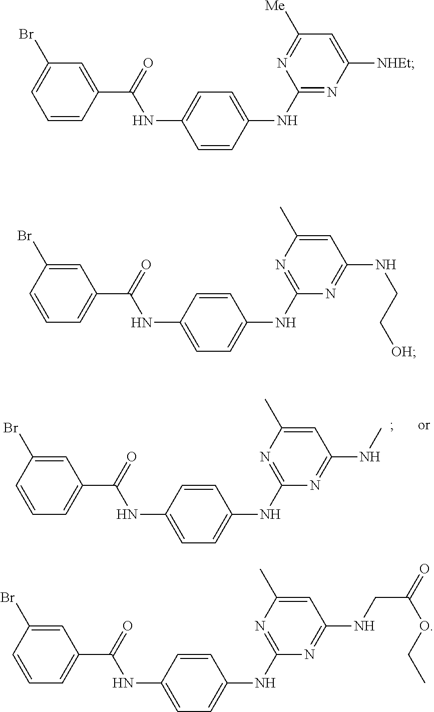

In more particular embodiments that may be mentioned herein, the compound of formula I, or a salt or solvate thereof, may be selected from the list:

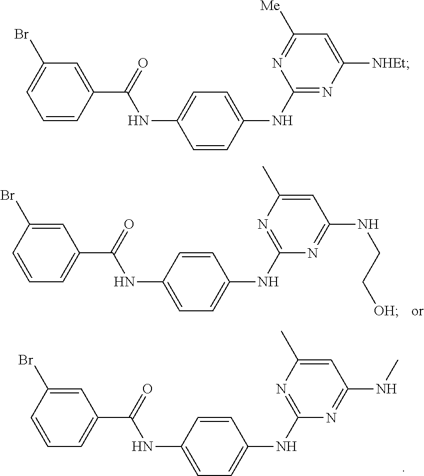

In the aspects and embodiments disclosed herein, the bacterial infection may be presented in the form of one or more of a respiratory, a skin or a soft tissue disorder. For example, the bacterial infection may be one or more of the group selected from a pulmonary infection, amyotrophic lateral sclerosis and bronchiectasis.

The compound of formula I as defined hereinbefore or a salt or a solvate thereof, may be used in combination with another therapeutic agent, or a salt or solvate thereof to provide a synergistic effect. Thus, the fourth to sixth aspects of the invention relate respectively to:
(A) use of a compound of formula I as defined hereinbefore or a salt or a solvate thereof, and another therapeutic agent, or a salt or solvate thereof, for the preparation of a medicament for the treatment of a bacterial infection caused by Mycobacterium abscessus, wherein the compound of formula I is administered sequentially, simultaneously or concomitantly with the other therapeutic agent;
(B) a compound of formula I, as defined hereinbefore or a salt or a solvate thereof, for use in the treatment of a bacterial infection caused by Mycobacterium abscessus, wherein the compound of formula I is administered sequentially, simultaneously or concomitantly with another therapeutic agent, or a salt or solvate thereof; and
(C) a method of treatment of a bacterial infection caused by Mycobacterium abscessus, which method comprises the administration of a pharmaceutically effective amount of a compound of formula I as defined hereinbefore or a salt or a solvate thereof, and another therapeutic agent, or a salt or solvate thereof, to a patient in need of such treatment.

When used herein, the term “another therapeutic agent” includes references to one or more (e.g. one) therapeutic agents (e.g. one therapeutic agent) that are known to be useful for (e.g. that are known to be effective in) the treatment of a bacterial infection caused by Mycobacterium abscessus.

When used herein, the term “administered sequentially, simultaneously or concomitantly” includes references to:
administration of separate pharmaceutical formulations (one containing the compound of formula I and one or more others containing the one or more other therapeutic agents); and
administration of a single pharmaceutical formulation containing the compound of formula I and the other therapeutic agent(s).

In said aspects of the invention, the another therapeutic agent may be selected from one or more of the group consisting of an oral antimicrobial agent, amikacin, cefocetin, clarithromycin, clofazimine, and imipenem. For example, the another therapeutic agent may be clofazimine.

As will be appreciated, the a compound of formula I as hereinbefore or a salt or a solvate thereof will be provided for use as a pharmaceutical composition. Thus, in a seventh aspect of the invention there is provided a pharmaceutical composition comprising a compound of formula I as defined hereinbefore or a salt or a solvate thereof in combination with one or more of a pharmaceutically acceptable adjuvant, diluent or carrier.

Compounds of formula I may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

Compounds of formula I will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of compound of formula I in any pharmaceutical formulation used in accordance with the present invention will depend on various factors, such as the severity of the condition to be treated, the particular patient to be treated, as well as the compound(s) which is/are employed. In any event, the amount of compound of formula I in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, compounds of formula I may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

As noted above, the compound of formula I or a salt or a solvate thereof, can be combined with a further therapeutic agent. Thus, in an eighth aspect of the invention there is provided a pharmaceutical composition comprising a compound of formula I as defined hereinbefore or a salt or a solvate thereof, and another therapeutic agent, or a salt or solvate thereof, wherein the another therapeutic agent is selected from one or more of the group consisting of an oral antimicrobial agent, amikacin, cefocetin, clarithromycin, clofazimine, and imipenem, optionally wherein the another therapeutic agent is clofazimine.

The combination product described above provides for the administration of component (A) in conjunction with component (B), and may thus be presented either as separate formulations, wherein at least one of those formulations comprises component (A) and at least one comprises component (B), or may be presented (i.e. formulated) as a combined preparation (i.e. presented as a single formulation including component (A) and component (B)).

Thus, there is further provided:
(I) a pharmaceutical formulation including a compound of formula I, as hereinbefore defined and another therapeutic agent, in admixture with a pharmaceutically-acceptable adjuvant, diluent or carrier (which formulation is hereinafter referred to as a “combined preparation”); and
(II) a kit of parts comprising components:
   (i) a pharmaceutical formulation including a compound of formula I, as hereinbefore defined, in admixture with a pharmaceutically-acceptable adjuvant, diluent or carrier; and
   (ii) a pharmaceutical formulation including another therapeutic agent, in admixture with a pharmaceutically-acceptable adjuvant, diluent or carrier, which components (i) and (ii) are each provided in a form that is suitable for administration in conjunction with the other.

Component (i) of the kit of parts is thus component (A) in admixture with a pharmaceutically-acceptable adjuvant, diluent or carrier. Similarly, component (ii) is component (B) in admixture with a pharmaceutically-acceptable adjuvant, diluent or carrier.

It will be appreciated that the combination products discussed above may be formulated in line with the discussion of the seventh aspect of the invention.

The aspects of the invention described herein (e.g. the above-mentioned compounds, combinations, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, or may have other useful pharmacological properties over, similar compounds, combinations, methods (treatments) or uses known in the prior art for use in the treatment of those conditions or otherwise.

Further aspects and embodiments of the invention will now be discussed by reference to the following non-limiting examples.

### EXAMPLES

### Materials

DNase I and MgCl₂ were purchased from Thermo Fischer, USA. Middlebrook 7H9 medium, CAMH broth, and Middlebrook albumin-dextrose-catalase (ADC) were purchased from BD Difco. Glycerol was purchased from Fisher Scientific. Tween 80 was purchased from Sigma-Aldrich. EDTA-free protease inhibitor cocktail was purchased from Roche-USA. CellTiter-glow and Bac titer glo were purchased from promega. 3-(N-morpholino) propanesulfonic acid (MOPS), ADP, NADH, lysozyme, KH₂PO₄. Clofazamine and Amikacin were purchased from Sigma-Aldrich. BCA kit was purchased from Pierce.

The M. abscessus subsp. abscessus ATCC 19977, M. bolletii, M. massiliense strain as well the clinical isolate M. abscessus Bamboo from a patient with amyotrophic lateral sclerosis and bronchiectasis were used. The latter belongs to M. abscessus subsp. abscessus and harbors the inactive, clarithromycin-sensitive erm(41) C28 sequevar (GenBank accession no. MVDX00000000) (Yee, M. et al., Genome Announc. 2017, 5, e00388-17).

### General Procedure for Culturing Mab Strains

All Mab strains were maintained in Middlebrook 7H9 medium supplemented with 0.2% (vol/vol) glycerol, 0.05% (vol/vol) Tween 80, and 10% (vol/vol) Middlebrook ADC. CAMH broth was used to culture Mab strains and was prepared according to the manufacturer's instructions.

### Example 1. Repurposing Approach for the GaMF1 Anti-TB Compound

The mycobacterial F₁F₀-ATP synthase consists of its proton translocation F₀ sector (a:C₉), which is connected by the central, rotating γ.ε and the peripheral stalk subunits b:b′:δ to the catalytic α₃:β₃ sector, in which ATP is formed (Kamariah, N. et al., J. Struct. Biol. 2019, 207, 199-208; Kamariah, N, et al., Prog. Biophys. Mol. Biol. 2020, 152, 64-73; and Guo, H. et al., Nature 2021, 589, 143-147). In contrast to nonmycobacterial F₁F₀-ATP synthase, the mycobacterial F-ATP synthase subunits α, δ, and γ contain a C-terminal elongation (Ragunathan, P. et al., J. Biol. Chem. 2017, 292, 11262-11279), inserted domain (Kamariah, N. et al., J. Struct. Biol. 2019, 207, 199-208), or an extra loop of 12 to 14 amino acids (Hotra, A. et al., FEBS J. 2016, 283, 1947-1961), respectively. These add-ons are essential for regulation (Kamariah, N, et al., Prog. Biophys. Mol. Biol. 2020, 152, 64-73; Guo, H. et al., Nature 2021, 589, 143-147; Ragunathan, P. et al., J. Biol. Chem. 2017, 292, 11262-11279; and Wong, C.-F. & Gruber, G., Antimicrob. Agents Chemother. 2020, 64, e01568-20) or ATP synthesis (Ragunathan, P. et al., J. Biol. Chem. 2017, 292, 11262-11279; Hotra, A. et al., FEBS J. 2016, 283, 1947-1961; and Harikishore, A. et al., Antibiotics 2021, 10, 1456). For example, the mycobacterial extra loop of subunit γ provides a failsafe device (Montgomerya, M. G. et al., Proc. Natl. Acad. Sci. U.S.A 2021, 118, e2111899118) by which one of its aspartate residues forms a salt bridge with an arginine residue of the peripheral stalk subunit b′ during rotation, when subunit g comes into proximity of subunit b′ (Montgomerya, M. G. et al., Proc. Natl. Acad. Sci. U.S.A 2021, 118, e2111899118). These novel mycobacterial features of subunits α and γ are targets for specific and novel inhibitors (Harikishore, A. et al., Antibiotics 2021, 10, 1456; and Hotra, A. et al., Angew. Chem. Int. Ed. Engl. 2020, 59, 13295-13304). GaMF1, a bactericidal anti-TB compound that targets the extra loop of the M. tuberculosis γ subunit, represents such a novel mycobacterial F-ATP synthase inhibitor, which has a c log P of 4.37 and good metabolic stability in mouse liver microsomes and does not inhibit growth of Gram-positive and Gram-negative bacteria like Staphylococcus aureus and Escherichia coli (Hotra, A. et al., Angew. Chem. Int. Ed. Engl. 2020, 59, 13295-13304).

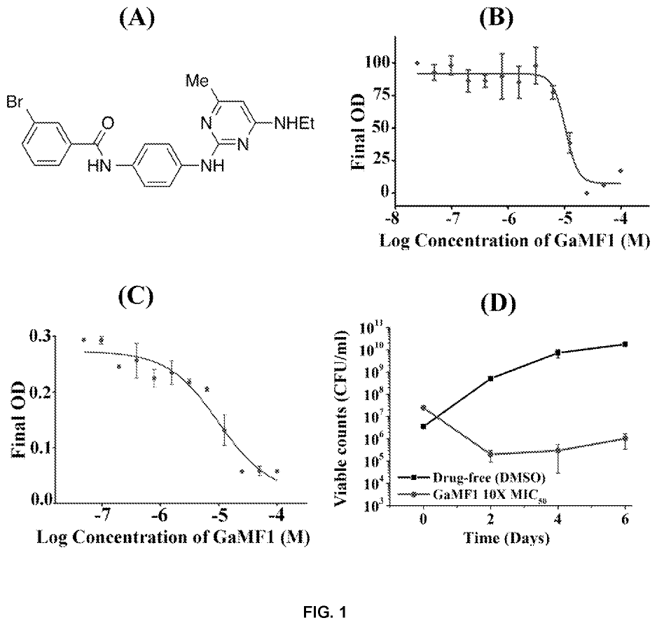

A repurposing approach was used for the GaMF1 anti-TB compound (FIG. 1A) and Mab's (M. abscessus subsp. abscessus) susceptibility to the compound in complete Middlebrook 7H9 broth, and M. abscessus's susceptibility to the compound in cation-adjusted Mueller-Hinton (CAMH) medium (Pang, H. et al., Int. J. Clin. Exp. Med. 2015, 8, 15423-15431; and Low, J. L. et al., Front. Microbiol. 2017, 8, 1539) and in complete Middlebrook 7H9 broth (Broda, A. et al., J. Clin. Microbiol. 2013, 51, 217-223) using the type strain M. abscessus subsp. abscessus ATCC 19977 as the test organism were studied. GaMF1 was synthesized according to literature (Hotra, A. et al., Angew. Chem. Int. Ed. Engl. 2020, 59, 13295-13304).

Determination of minimum inhibitory concentration (MIC) The growth inhibition dose-response assay was carried out using the broth microdilution method as described previously (Moreira, W., Aziz, D. B. & Dick, T., Front. Microbiol. 2016, 7, 199). The MIC50 reported represents the concentration that inhibits 50% of growth compared to the untreated culture.

### Results and Discussion

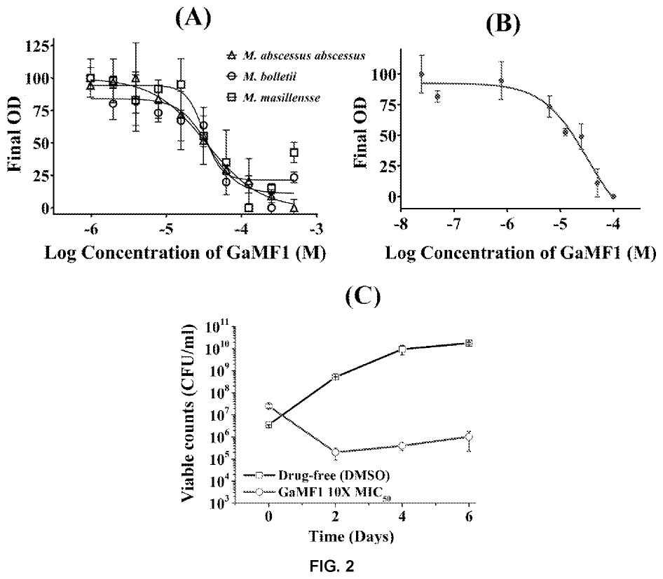

GaMF1 displayed potency with a MIC required to inhibit the growth of 50% of M. abscessus (MIC50) of 13±2.1 μM (FIG. 1B) and 33±4.7 M (FIG. 2A), which are even better than the MIC50 values of the first line drugs cefocetin (43 μM), amikacin (44 μM) and amikacin (360 UM) (Sarathy, J. P. et al., Antimicrob. Agents Chemother. 2020, 64, e02404). GaMF1 also inhibited 50% growth of the two additional subspecies M. bolletii at 36.2 μM and M. massiliense at 35.8 UM (FIG. 2A). These values are also comparable to the ones determined for GaMF1 in M. tuberculosis (33 UM) and M. bovis Calmette-Guérin (BCG, 17 μM, Hotra, A. et al., Angew. Chem. Int. Ed. Engl. 2020, 59, 13295-13304), and are in the range of the clinically used anti-M. abscessus drugs cefoxitin (38 UM, Ferro, B. E. et al., Antimicrob. Agents Chemother. 2016, 60, 6374-6376), amikacin (55 UM, Ferro, B. E. et al., Antimicrob. Agents Chemother. 2016, 60, 6374-6376), and imipenem (14 UM, Brown-Elliott, B. A. et al., J. Clin. Microbiol. 2016, 54, 1586-1592). While the F-ATP synthase inhibitors bedaquiline (BDQ) and TBAJ876 reveal a 100× higher potency against M. tuberculosis than M. abscessus (Sarathy, J. P. et al., Antimicrob. Agents Chemother. 2020, 64, e02404-19), GaMF1 retains similar efficacy in cell growth inhibition and killing over a wide spectrum of mycobacteria.

### Example 2. GaMF1 Susceptibility

We determined whether GaMF1 susceptibility is retained against the clinical isolate M. abscessus subsp. abscessus Bamboo (Yee, M. et al., Genome Announc. 2017, 5, e00388-17). MIC50 was determined by following the protocol in Example 1.

### Results and Discussion

The MIC50 determined in CAMH medium and Middlebrook 7H9 broth was 10±1.9 UM (FIG. 1C) and 33±3.2 μM (FIG. 2B), respectively, similar to the values determined for the M. abscessus type strain M. abscessus subsp. abscessus ATCC 19977.

### Example 3. Bactericidal activity of GaMF1

To determine whether GaMF1 is bactericidal against M. abscessus, we measured the survival of the type strain upon drug exposure. MIC50 was determined by following the protocol in Example 1.

### Results and Discussion

GaMF1 was bactericidal against M. abscessus at 10-fold its MIC50 in both CAMH medium (FIG. 1D) and 7H9 (FIG. 2C).

### Example 4. Ability of GaMF1 to Inhibit Mycobacterial F-ATP Synthase

The ability of GaMF1 to inhibit mycobacterial F-ATP synthase was evaluated using an intracellular ATP synthesis assay (Lechartier, B. & Cole, S. T., Antimicrob. Agents Chemother. 2015, 59, 4457-4463) and IMVs of M. abscessus (Hotra, A. et al., Angew. Chem. Int. Ed. Engl. 2020, 59, 13295-13304), which were prepared according to Hotra et al. (Hotra, A. et al., Angew. Chem. Int. Ed. Engl. 2020, 59, 13295-13304).

Preparation of Inverted Membrane Vesicles from Mab

In order to purify IMVs of Mab for ATP synthesis assays, cells were grown overnight at 37° C. in 7H9 supplemented with 10% Middlebrook albumin-dextrose-catalase (ADC), 0.5% glycerol and 0.05% Tween 80 until they reached an OD₆₀₀ value of 0.4. The culture was expanded in 200 ml supplemented 7H9 and grown in a roller bottle (2 rpm) until OD₆₀₀=0.4. This culture was used to inoculate a 5 l culture that was grown overnight in roller bottles until an OD₆₀₀=0.4. About 5 g (wet weight) of Mab was resuspended in 20 ml membrane preparation buffer (50 mM MOPS, 2 mM MgCl₂, pH 7.5) containing EDTA-free protease inhibitor cocktail (1 tablet in 20 ml buffer) and 1.2 mg/ml lysozyme. The suspension was stirred at room temperature for 45 min and additionally supplemented with 300 μl 1 M MgCl₂ and 50 μl DNase I, and continued stirring for another 15 min at room temperature. All subsequent steps were performed on ice. Cells were broken by three passages through an ice precooled Model M-110L Microfluidizer processor (M-110L) at 18,000 psi. The suspension containing lysed cells was centrifuged at 4,200×g at 4° C. for 20 min. The supernatant containing membrane fraction was further subjected to ultracentrifugation 45,000×g at 4° C. for 1 h. The supernatant was discarded, and the precipitated membrane fraction was resuspended in membrane preparation buffer containing 15% glycerol, aliquoted, snap frozen and stored at −80° C. The concentrations of the proteins in the vesicles were determined by the BCA method (Smith, P. K et al., Anal Biochem. 1985, 150, 76-85).

### ATP Synthesis Assay of IMVs

ATP synthesis was measured in flat bottom white microtiter 96 well plates (Corning USA). The reaction mixture was made in assay buffer (50 mM MOPS, pH 7.5, 10 mM MgCl₂) containing 10 μM ADP, 250 μM Pi and 1 mM NADH. The concentration of Pi was adjusted by adding 100 mM KH₂PO₄ salt dissolved in the assay buffer. ATP synthesis was started by adding inverted vesicles of Mab to a final protein concentration of 5 μg/ml. The reaction mixture was incubated at room temperature for 30 min before addition of 50 μl of the CellTiter-glow reagent, and the mixture was incubated for another 10 min in dark at room temperature. Emitted luminescence, which is correlated to the synthesized ATP, was measured by a Tecan plate reader Infinite 200 Pro (Tecan USA), using the following parameters: luminescence, integration time of 500 ms, and no attenuation.

### Whole Cell ATP Measurement

Whole cell ATP synthesis assays were carried out as described previously (Hotra, A. et al., Angew. Chem. Int. Ed. Engl. 2020, 59, 13295) in 96 well plates.

### Half-Maximal Inhibitory Concentration (IC₅₀)

100 μl of complete 7H9 medium were filled in each well of clear 96-well flat-bottom Costar cell culture plates (Corning). GaMF1 was added to the first well in each row to create two times the desired highest final concentration. Subsequently, a 16-point 2-fold serial dilution was carried out starting from the first well in each row. M. abscessus subsp abscessus, which was grown to mid-exponential phase, was diluted to an optical density at 600 nm (OD₆₀₀) of 0.1; 100 μl of the diluted culture was added to each well to create a final OD₆₀₀ of 0.05 in all the wells. The plates were incubated at 37° C. for 6-8 h.

At the end of the incubation period, the samples were measured for their intracellular ATP content by adding the BacTiter-Glo microbial cell viability assay (Promega), which was carried out according to the manufacturer's instructions as described previously (Wu, M.-L., Gengenbacher, M. & Dick, T., Front Microbiol. 2016, 7, 947). 50 μl of bacterial sample was mixed with 50 μl of the BacTiter-Glo reagent in each well of an opaque, white, 96-well, flat-bottom Nunc plate. Luminescence was measured with a Tecan Infinite Pro 200 plate reader after 10 min of incubation of the plate in the dark at room temperature. The background luminescence reading was subtracted from the luminescence readings of all the samples. The amount of ATP content is directly proportional to the relative luminescence units. The graph of the results was made using GraphPad Prism 8 software. IC₅₀ corresponds to 50% reduction of luminescence values when compared to drug-free condition.

### Results and Discussion

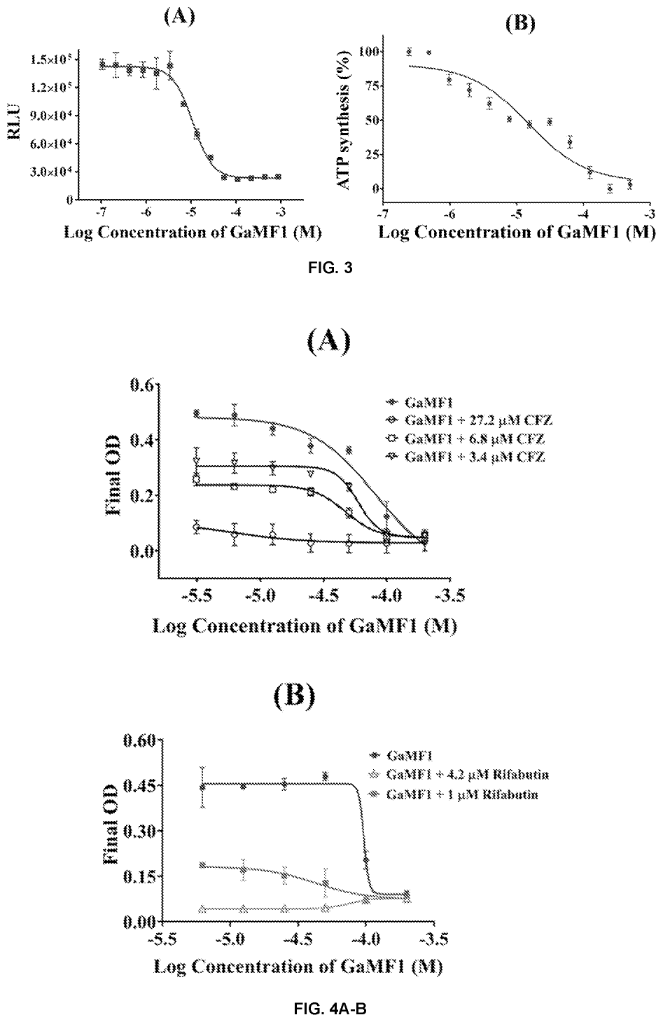

As displayed in FIG. 3A, compound GaMF1 depleted ATP synthesis within the cell with a IC₅₀ of 10±0.9 μM and depleted NADH-driven ATP synthesis of M. abscessus IMVs with an IC₅₀ of 13±2.5 μM (FIG. 3B), suggesting that growth inhibition of the pathogen is caused by the inhibition of the F-ATP synthase.

TABLE 1
GaMF1 and its effects to cell growth (MIC₅₀) and ATP synthesis (IC₅₀).

| | | | | MIC₅₀ [μM] | |
|---|---|---|---|---|---|
| | | Commercially | Synthesis | CAMH | |
| Name | Structure | available | known | medium | IC₅₀ [μM] |
| GaMF1 | 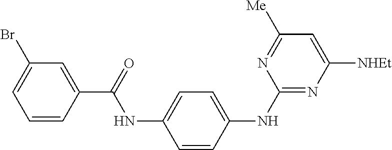 | Yes | Hotra, A. et al., Angew. Chem. Int. Ed. Engl. 2020, 59, 13295- 13304 | 13 | 10 |

### Example 5. Growth Inhibition Activity of GaMF1 Combined with CFZ

Since treatment of M. abscessus infections requires drug combinations, we measured the growth inhibition activity of GaMF1 combined with CFZ, proposed to compete with the mycobacterial specific electron acceptor menaquinone for its reduction by the NDH-2 complex (Lechartier, B. & Cole, S. T., Antimicrob. Agents Chemother. 2015, 59, 4457-4463). CFZ has been recently shown to be active against M. abscessus and is used clinically as a repurposed drug. MIC50 was determined by following the protocol in Example 1.

### Results and Discussion

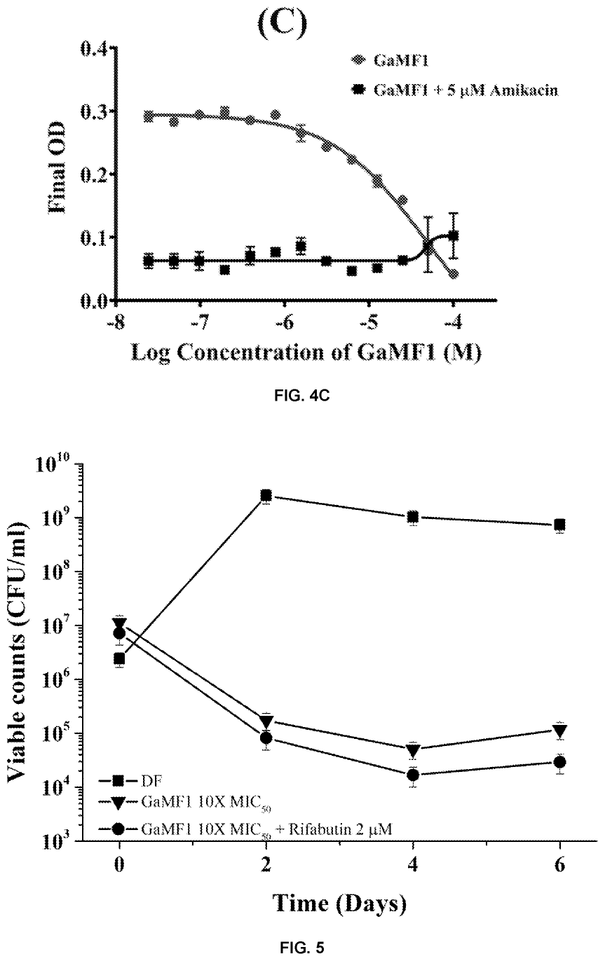

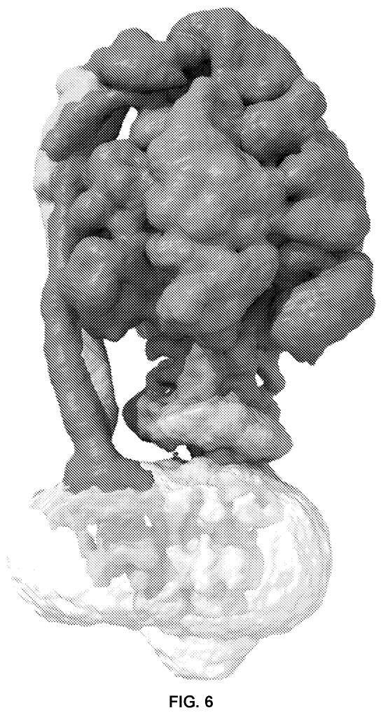

As revealed in FIG. 4, the combination of 3.4 μM CFZ or 6.8 μM (MIC50 of CFZ, Ferro, B. E. et al., Antimicrob. Agents Chemother. 2016, 60, 1097-1105) with GaMF1 increased the potency of M. abscessus cell inhibition in 7H9 medium (FIG. 4A). When GaMF1 was combined with M. abscessus's RNA polymerase inhibitor rifabutin (MIC of 1 to 4 μM) (FIG. 4B) or the anti-M. abscessus antibiotic amikacin (5 μM, FIG. 4C), a drastic reduction of cell growth was observed.

### Example 6. GaMF1 and Rifabutin Killing Potency

### GaMF1 and Rifabutin Killing Potency Studies

M. abscessus culture were grown to exponential phase and diluted to an OD₆₀₀ of 0.005 and aliquoted onto T-25 mm² tissue culture flasks. GaMF1 along with Rifabutin were dispensed into each flask and were incubated at 37° C. with shaking at 180 rpm for 5 days. Around 10 μl of culture was taken out from each flask, followed by the serial dilution with PBS. 25 μl of culture of respective dilutions were plated on each quadrant of 7H10 agar plate. The agar plates were incubated at 37° C. for three days. Bacterial viability was determined by counting the colony-forming units (CFU) on the plates.

### Results and Discussion

Considering the increased anti-M. abscessus activity in the GaMF1-rifabutin combination and rifabutin's bactericidal activity (Aziz, D. B. et al., Antimicrob. Agents Chemother. 2017, 61, e00155-17; and Johansen, M. D. et al., Antimicrob. Agents Chemother. 2020, 64, e00363-20), the combinatory GaMF1-rifabution effect in M. abscessus killing was shown to be only marginal (FIG. 5).

In conclusion, this study has shown that GaMF1 is active in cell growth inhibition and killing of M. abscessus, similar to the compound's activity against M. tuberculosis. The identification of GaMF1 as a novel M. abscessus inhibitor expands the poorly populated compound pipeline against this difficult-to-cure opportunistic NTM pathogen. By targeting M. abscessus's F-ATP synthase, GaMF1 depletes cellular ATP, essential for cell wall formation, replication, ana- and catabolic processes, and ATP-dependent efflux pumps. Inhibition of the latter could impede drug flux, which has emerged as an important determinant of drug resistance of BDQ and CFZ in M. abscessus. Finally, M. abscessus revealed an increased susceptibility of GaMF1 in combination with CFZ, rifabutin, or amikacin, which is a prerequisite for combinatory approaches to treat M. abscessus infections.

1. (canceled)

2. (canceled)

3. A method of treatment of a bacterial infection caused by Mycobacterium abscessus comprising the steps of providing a pharmaceutically effective amount of a compound of formula I, 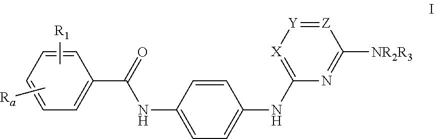
where:
R₁ and Rₐ each independently represent H, halogen, C₁-C₄ alkyl, C₁-C₄ alkoxy, or OH;
R₂ represents H, C₁-C₄ alkyl that is unsubstituted or substituted by a hydroxyl group, or
—CH₂CO₂Et;
R₃ represents H or C₁-C₄ alkyl;
X, Y and Z each independently represent CR₄ or N;
each R₄ independently represents H, halogen, C₁-C₄ alkyl, or C₁-C₄ alkoxy, or a salt or solvate thereof, to a subject in need thereof.

4. The method according to claim 3, wherein the compound of formula I is one in which R₂ represents H, C₁-C₄ alkyl that is unsubstituted or substituted by a hydroxyl group.

5. The method according to claim 3, wherein R₃ represents H, CH₃ or CH₂CH₃.

6. The method according to claim 3, wherein Rₐ is H and R₁ is Br.

7. The method according to claim 3, wherein X is N.

8. The method according to claim 3, wherein Z is N.

9. The method according to claim 3, wherein Y is C—CH₃.

10. The method according to claim 3, wherein the compound of formula I, or a salt or solvate thereof, is selected from the list:

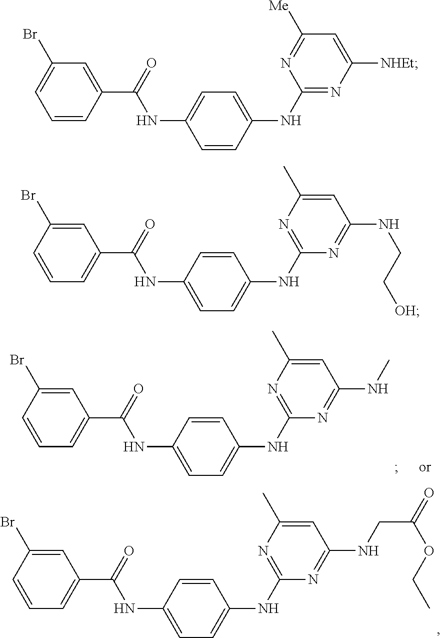

11. The method according to claim 3, wherein the bacterial infection is presented in the form of one or more of a respiratory, a skin or a soft tissue disorder (e.g. one or more of the group selected from a pulmonary infection, amyotrophic lateral sclerosis and bronchiectasis).

12. (canceled)

13. (canceled)

14. A method of treatment of a bacterial infection caused by Mycobacterium abscessus, which method comprises the administration of a pharmaceutically effective amount of a compound of formula I as defined in claim 3 or a salt or a solvate thereof, and another therapeutic agent, or a salt or solvate thereof, to a patient in need of such treatment.

15. The method according to claim 14, wherein the another therapeutic agent is selected from one or more of the group consisting of an oral antimicrobial agent, amikacin, cefocetin, clarithromycin, clofazimine, and imipenem.

16. A pharmaceutical composition comprising a compound of formula I as defined in claim 3 or a salt or a solvate thereof, and another therapeutic agent, or a salt or solvate thereof, wherein the another therapeutic agent is selected from one or more of the group consisting of an oral antimicrobial agent, amikacin, cefocetin, clarithromycin, clofazimine, and imipenem.

17. The method according to claim 3, wherein the compound of formula I, or a salt or solvate thereof, is selected from the list:

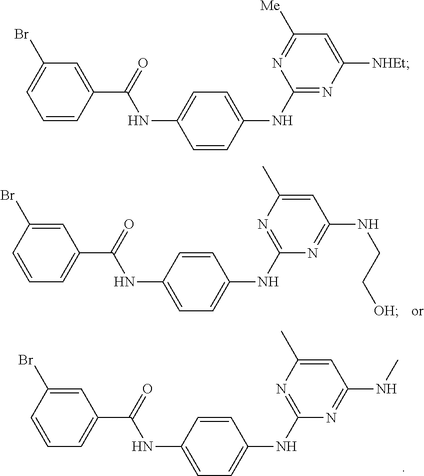

18. The method according to claim 15, wherein the another therapeutic agent is clofazimine.

19. The pharmaceutical composition according to claim 16, wherein the another therapeutic agent is clofazimine.
